(19) United States
(12) Patent Application Publication
(10) Pub. No.: US 2021/0156792 A1
(43) Pub. Date: May 27, 2021

(54) OCCULT BLOOD TESTING DEVICE

(71) Applicant: TAIWAN REDEYE BIOMEDICAL INC., HSINCHU CITY (TW)

(72) Inventors: SHUO-TING YAN, HSINCHU CITY (TW); I-HUA WANG, HSINCHU CITY (TW)

(21) Appl. No.: 16/691,922

(22) Filed: Nov. 22, 2019

(51) Int. Cl. G01N 21/27; G01N 33/72; G01N 21/25

(52) U.S. Cl. CPC G01N 21/27; G01N 2201/0221; G01N 21/255; G01N 33/726

(57) ABSTRACT

A handheld occult blood testing device is disclosed. The occult blood testing device includes a hemoglobin sensor, at least one elastic clip and a sleeve. The elastic clip is disposed on the surface of the hemoglobin sensor and the sleeve is sleeved over the hemoglobin sensor, wherein a plurality of through holes are disposed on the surface of the sleeve for the elastic clip to pass through and be exposed from the surface of the sleeve. The sleeve is movable relative to the hemoglobin sensor between a first position and a second position, wherein the sleeve at the first position does not press the elastic clip such that the elastic clips a filter unit to the hemoglobin sensor, and the sleeve at the second position compresses the elastic clip such that the elastic clip releases the filter unit on the hemoglobin sensor.

### FIELD OF THE INVENTION

The invention relates to an occult blood testing device, more particularly, to an occult blood testing device that is convenient for user to use and clean.

### BACKGROUND

With the changes in people's diet and life habits, colorectal cancer has become one of the most common cancer types diagnosed. In Taiwan, colon cancer rate has topped the cancer ranking for eleven consecutive years and is also the highest in the world. The incidence rate and death rate of colon cancer patients are respectively 18.6% and 20.1% in China, which both topped the rankings in the world. The incidence rate for colon cancer is 8% out of all cancer patients, which ranks fourth and the death rate ranks second in USA.

Testing for fecal occult blood is one of the methods to detect colon cancer at early stage. Conventional stool occult blood test requires collecting fecal specimen, sending it to the hospital for testing and waiting for the test result, which not only is inconvenient for the patient, the inability of testing-at-anytime also prohibits self-management of the patient for long-term health monitoring.

Moreover, the specimen collected for conventional testing is about 1 gram, but only 6 milligrams is used by the medical unit for testing. However, occult blood in the stool is not distributed evenly and so false negative result is easily concluded from the testing when the occult blood was not collected in the specimen. In addition, the bleeding of tumor or polyp is periodic, not continuous, and so it is possible that the tumor or the polyp did not bleed on the day of or the day before the collection of stool specimen, causing the test result to be false negative. Hence, improving the accuracy and convenience of stool occult blood test is an important factor for early diagnosis and treatment of colon cancer to lower the death rate.

### SUMMARY

An objective of the invention is to provide an occult blood testing device that is convenient for user to clean after taking the test and is capable of preventing solid residues in the test solution from contaminating the testing device or the user.

Another objective of the invention is to provide an occult blood testing device having at least one elastic clip disposed on the surface of a hemoglobin sensor for clipping and releasing a filter unit on the hemoglobin sensor, so the user is able to clean the hemoglobin sensor easily.

Yet another objective of the invention is to provide an occult blood testing device having a sleeve sleeved on the hemoglobin sensor, wherein the sleeve is movable along the hemoglobin sensor between a first position and a second position. The sleeve at the first position does not press the elastic clip and so the elastic clip clips a filter unit on the hemoglobin sensor. The sleeve at the second position compresses the elastic clip and forces the elastic clip to release the filter unit on the hemoglobin sensor.

To achieve the objectives mentioned above, the present invention provides an occult blood testing device, which includes a hemoglobin sensor having a probe unit, at least one light emitting unit for emitting at least one incident light beam, at least one light sensing unit for receiving at least one detection light beam, at least one elastic clip, and a sleeve. The incident light beam is aimed towards a direction of the probe unit and at a test solution to produce the detection light beam. The elastic clip is disposed on a surface of the hemoglobin sensor and includes a push portion and a clip portion, wherein the clip portion clips and holds a filter unit to the hemoglobin sensor. The sleeve is sleeved over the hemoglobin sensor and is movable relative to the hemoglobin sensor between a first position and a second position, wherein when the sleeve is at the first position, the push portion of the elastic clip is not pressed and the clip portion of the elastic clip is rested on the probe unit and clips the filter unit, and when the sleeve is at the second position, the push portion of the elastic clip is compressed to force the clip portion of the elastic clip to move away from the probe unit and release the filter unit.

In one embodiment of the invention, the sleeve includes a plurality of through holes disposed thereon and the push portion of the elastic clip is positioned inside the through hole of the sleeve.

In one embodiment of the invention, the elastic clip includes a lug, the probe unit includes a plurality of connect recesses disposed on a surface of the probe unit, and the lug of the elastic clip is positioned inside the connect recess of the probe unit.

In one embodiment of the invention, a plurality of clip recesses are disposed on the surface of the probe unit and the clip portion of the elastic clip is positioned inside the clip recess of the probe unit.

In one embodiment of the invention, the elastic clip includes an inner surface and an outer surface, the clip portion and the lug are disposed on and protruding from the inner surface, and the push portion is an arc structure curved in a direction from the inner surface to the outer surface.

In one embodiment of the invention, the elastic clip includes a rotating axle disposed on the lug, the probe unit includes a plurality of protrusions disposed on the side of the connect recess, and the rotating axle of the elastic clip is positioned between two of the protrusions.

In one embodiment of the invention, the occult blood testing device further includes an elastic unit disposed between the sleeve and the hemoglobin sensor, and the sleeve at the second position compresses the elastic unit.

In one embodiment of the invention, the hemoglobin sensor includes a flange disposed on an outer surface of the hemoglobin sensor, the sleeve includes a protruding unit disposed on an inner surface of the sleeve, and two ends of the elastic unit are respectively connected to the flange of the hemoglobin sensor and the protruding unit of the sleeve.

In one embodiment of the invention, the sleeve includes an opening disposed at one end of the sleeve and a plurality of indentations disposed around the opening, wherein the opening and the indentations are in fluid communication with an accommodating space in the sleeve, the probe unit is positioned inside the opening of the sleeve, and the elastic clip is positioned inside the indentation of the sleeve.

In one embodiment of the invention, the probe unit includes at least one opening and a test space, wherein the opening is disposed at one end of the probe unit and is fluidly connected to the test space.

### BRIEF DESCRIPTION OF THE DRAWINGS

The structure as well as preferred modes of use, further objects, and advantages of this invention will be best understood by referring to the following detailed description of some illustrative embodiments in conjunction with the accompanying drawings, in which:

FIG. 1 is an exploded view of an occult blood testing device according to an embodiment of the invention.

FIGS. 2A and 2B are respectively a sectional view and a schematic diagram of an occult blood testing device according to an embodiment of the invention.

FIGS. 3A and 3B are respectively a sectional view and a schematic diagram of an occult blood testing device operated in a first position and with a sleeved filter unit according to an embodiment of the invention.

FIGS. 4A and 4B are respectively a sectional view and a schematic diagram of an occult blood testing device operated in a first position and with a clipped filter unit according to an embodiment of the invention.

FIGS. 5A and 5B are respectively a sectional view and a schematic diagram of an occult blood testing device operated in a second position to remove a filter unit according to an embodiment of the invention.

### DETAILED DESCRIPTION OF THE PREFERRED EMBODIMENTS

The term “solution” or “test solution” in the invention refers to solution excreted or produced directly by human or animal, liquid of the solution, or solution of other solvent; for example, solution in the toilet with excreta such as feces, urine, and/or phlegm therein. The term “occult blood” refers to human or animal blood in the excreta, especially blood that is invisible to human eyes.

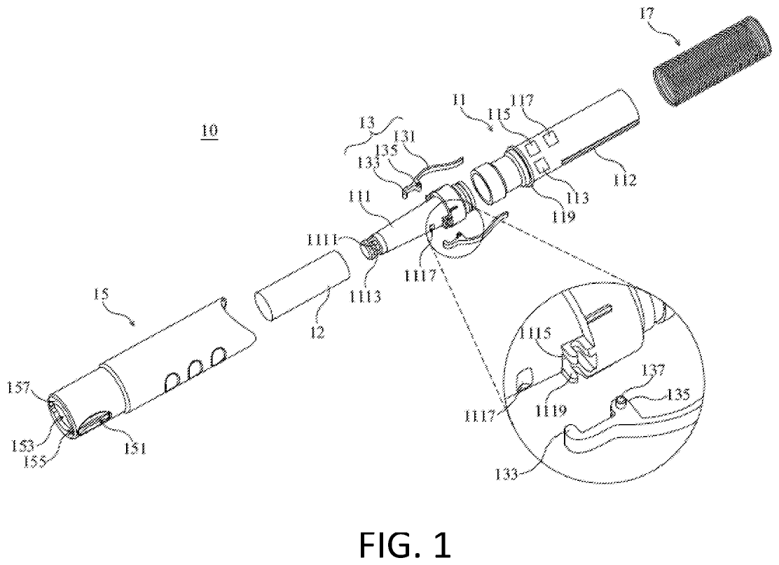

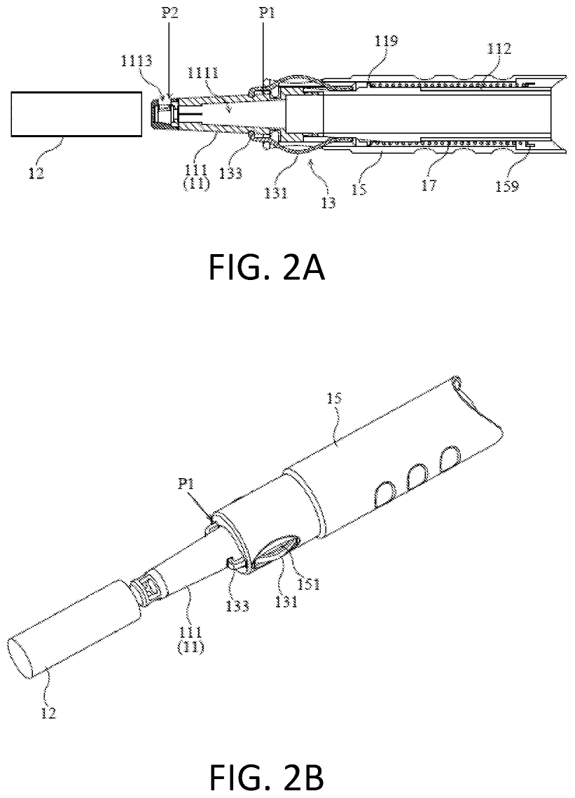

FIGS. 1, 2A and 2B are, respectively, an exploded view, a sectional view and a schematic diagram of an occult blood testing device according to a preferred embodiment of the invention. The occult blood testing device 10 includes a hemoglobin sensor 11, at least one elastic clip 13 and a sleeve 15, wherein the hemoglobin sensor 11 detects whether blood exists in excreta like feces, urine and/or phlegm through optical mechanism.

The hemoglobin sensor includes a probe unit 111, at least one light emitting unit 113, at least one light sensing unit 115, and a processing unit 117, wherein a test solution can be stored/placed in the probe unit 111. Specifically, the probe unit 111 is a hollow tube with a test space 1111 therein, and an opening 1113 is disposed at one end (front end) of the probe unit 111, wherein the opening 1113 is fluidly coupled/connected to the test space 1111 in the probe unit 111. When using the occult blood testing device 10, the probe unit 111 is inserted into the test solution and the test solution flows through the opening 1113 and enters the test space 1111 of the probe unit 111.

The light emitting unit 113 is used to emit at least one incident light beam, which is aimed towards the direction of the probe unit 111 and at the test solution, and subsequently at least one detection light beam is produced. The light emitting unit 113 includes, for example, at least one light emitting diode (LED). The light sensing unit 115 is used to receive the detection light beam and generate a detection signal.

In particular, the incident light beam is aimed at the test solution in the test space 1111 of the probe unit 111, wherein the test solution may absorb a part of the incident light beam and thereby producing the detection light beam having specific wavelength. The light sensing unit can be a phototransducer and transforms the received detection light beam to the detection signal in the form of electrical signal.

The processing unit 117 is electrically connected to the light sensing unit 115 and receives the detection signal from the light sensing unit 155 to analyze the detection signal and generate a detection result data. In practical application, the light emitting unit 113 is electrically connected to an operating interface such as buttons or touch screen, and through the operating interface, user can control the light emitting unit 113 to emit the plurality of incident light beams.

In one embodiment of the invention, the hemoglobin sensor 11 includes a connecting tube 112, wherein the connecting tube 112 is a hollow tube and is connected to another end (back end) of the probe unit 111, and the light emitting unit 113, the light sensing unit 115, and the processing unit 117 are disposed in the connecting tube 112.

In one embodiment of the invention, the elastic clip 13 is disposed on the surface of the hemoglobin sensor 11 and/or the probe unit 111 and includes a push portion 131 and a clip portion 133. More specifically, the elastic clip 13 is a long strip with an inner surface and an outer surface. The clip portion 133 is on the inner surface of the elastic clip and the push portion 131 is on the outer surface of the elastic clip 13. For example, the push portion 131 is an arc structure of the elastic clip 13 that is curved in a direction from the inner surface to the outer surface, and the clip portion 133 is a structure protruding from the inner surface of the elastic clip 13.

In one embodiment of the invention, the elastic clip 13 includes a lug 135 disposed on the inner surface of the elastic clip 13, wherein the clip portion 133 and the lug 135 protrude from the inner surface of the elastic clip 13. For example, the clip portion 133 is disposed at one end of the elastic clip 13, and the lug 135 is disposed at a middle region of the elastic clip 13.

On the surface of the hemoglobin sensor 11 and/or the probe unit 111, a plurality of connect recesses 1115 are disposed. The lug 135 of the elastic clip 13 is positioned/placed inside the connect recess 1115 on the surface of the probe unit 111, by which the elastic clip 13 is connected to the hemoglobin sensor 11 and/or the probe unit 111 to prevent the elastic clip 13 from moving or sliding relative to the hemoglobin sensor 11 and/or the probe unit 111.

In one embodiment of the invention, on a side of the connect recess 1115, a plurality of protrusions 1119 are disposed, wherein two adjacent protrusions 1119 form a protruded U-shaped structure on the surface of the probe unit 111, in which a recess is formed between the two adjacent protrusions 1119. Moreover, at least one rotating axel 137 is disposed on a side of the lug 135 of the elastic clip 13, wherein the rotating axel 137 is positioned in the recess between the two adjacent protrusions 1119. When a user presses the push portion 131 of the elastic clip 13, the clip portion 133 of the elastic clip 13 is pivoted by the rotating axel 137 acting as the axis, and depending on the user's action, the clip portion 133 of the elastic clip 13 rests on or moves away from the surface of the probe unit 111 to clip or release a filter unit 12 between the elastic clip 13 and the hemoglobin sensor 11.

In another embodiment of the invention, a plurality of clip recesses 1117 are disposed on the surface of the hemoglobin sensor 11 and/or the probe unit 111, wherein the clip recesses 1117 are on the surface of the probe unit 111 that is between the connect recess 1115 and the opening 1113. When the lug 135 of the elastic clip 13 is positioned/placed inside the connect recesses 1115, the clip portion 133 of the elastic clip 13 is positioned inside the clip recess 1117 of the probe unit 111. occult blood testing device

While using the occult blood testing device 10, user can put a filter unit 12 on the probe unit 111, wherein the filter unit 12 is, for example, a tubular filter with one open end and one closed end, and the open end of the filter unit 12 is clipped by the clip portion 133 of the elastic clip 13 to the surface or the clip recess 1117 of the probe unit 111. Two elastic clips 13 are shown in the figures of the invention, however, however, they are for exemplary purpose only, and the invention does not limit the number of the elastic clips. The occult blood testing device 10 with a single elastic clip 13 can also attach the filter unit 12 to the probe unit 111, and in other embodiments, the number of elastic clips can be greater than two.

The sleeve 15 is sleeved over the hemoglobin sensor 11 and/or the elastic clip 13 partially, wherein the sleeve 15 is a tube with two open and connecting ends, and there are a plurality of through holes 131 disposed on the surface of the sleeve 15. When the sleeve 15 is disposed on the hemoglobin sensor 11 and the elastic clip 13, the push portion 131 of the elastic clip 13 is positioned inside the through hole 151, wherein the push portion 131 passes through the through hole 151 and is exposed beyond the outer surface of the sleeve 15 as shown in FIGS. 2A and 2B.

At one end (front end) of the sleeve 15, an opening 153 and a plurality of recesses 155 are disposed, wherein the area of the opening 153 is slightly bigger than the sectional area of the probe unit 111, and the recesses 155 are disposed around and at the periphery of the opening 153. The opening 153 and the recesses 155 are in fluid communication to an accommodating space in the sleeve 15. When putting the sleeve 15 on the hemoglobin sensor 11, the probe unit 111 passes through the opening 153 of the sleeve 15, and the elastic clip 13 disposed on the surface of the probe unit 111 passes through the recesses 155 of the sleeve 15. Moreover, the front end of the sleeve 15 includes a block portion 157 disposed thereat, wherein the block portion 157 extends axially from the outer surface of the sleeve 15 and is at the periphery of the opening 153 and the recesses 155.

When using the occult blood testing device 10, the sleeve 15 is movable relative to the hemoglobin sensor 11 and/or the probe unit 111 between a first position P1 and a second position P2 as shown in FIGS. 2A and 2B. When the sleeve 15 is at the first position, the push portion 131 of the elastic clip 13 passes through and is exposed by the through hole 151 of the sleeve 15, such that the user can press the push portion 131 of the elastic clip 13 to control the clip portion 133 of the elastic clip 13 for clipping or releasing the filter unit 12. More particularly, the sleeve 15 at the first position P1 does not press the push portion 131 of the elastic clip 13, and so the clip portion 133 of the elastic clip 13 rests on the hemoglobin sensor 11 and/or the probe unit 111 and clips the filter unit 12 between the two. On the contrary, when the sleeve 15 is at the second position P2, the inner surface of the sleeve 15 presses against the push portion 131 of the elastic clip and the clip portion 133 of the elastic clip 13 is forced to leave or move away from the hemoglobin sensor 11 and/or the probe unit 111 and release the filter unit 12 between the two. In addition, during the displacement of the sleeve 15 from the first position P1 to the second position P2, the block portion 157 at the front end of the sleeve 15 pushes the filter unit 12 on the probe unit 111 to remove the filter unit 12 from the probe unit 111.

In another embodiment of the invention, the occult blood testing device 10 includes an elastic unit 17 like a compressed spring, wherein the elastic unit 17 is disposed between the hemoglobin sensor 11 and the sleeve 15, and the sleeve 15 at the second position P2 compresses the elastic unit 17.

In specific, the hemoglobin sensor 11 includes a flange 119 disposed on its outer surface, and the inner surface of the sleeve 15 has disposed thereon at least one protruding unit 159. As shown in FIG. 2A, one end of the elastic unit 17 is connected to the flange 119 of the hemoglobin sensor 11 and another end of the elastic unit 17 is connected to the protruding unit 159 of the sleeve 15. When the sleeve 15 moves towards the front end of the probe unit 111, the protruding unit 159 of the sleeve 151 and the flange 119 of the hemoglobin sensor 11 compress the elastic unit 17 thereinbetween

During the testing of occult blood with the occult blood testing device 10, the probe unit 111 is inserted into the test solution containing feces so as to allow the test solution to enter the test space 1111 through the opening of the probe unit 111. Since the solid residue of feces often exists in the test solution, the user must thoroughly clean the probe unit 111 that was in contact with the solid residue after the occult blood testing.

To solve the aforementioned issue, the filter unit 12 is disposed on or sleeved over the probe unit 111 before testing to prevent the solid residue in the test solution from entering the test space 1111 of the probe unit 111. After the test, the user simply removes the filter unit 12 from the probe unit 111 and rinses/washes the probe unit 111 with running water to clean the occult blood testing device 10.

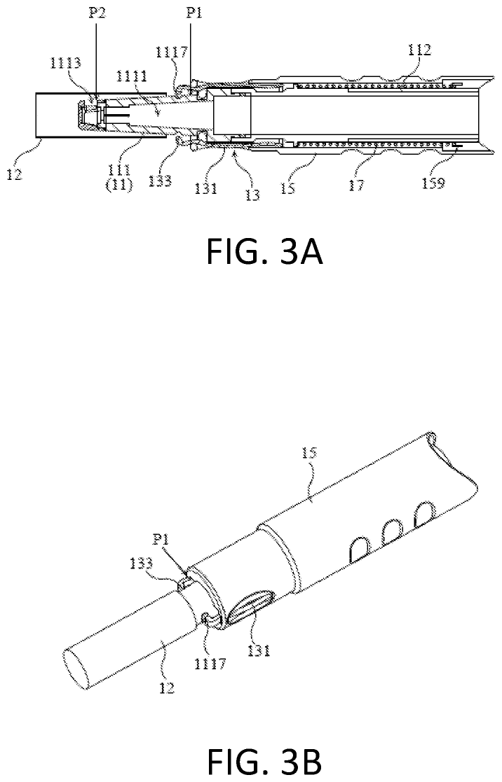

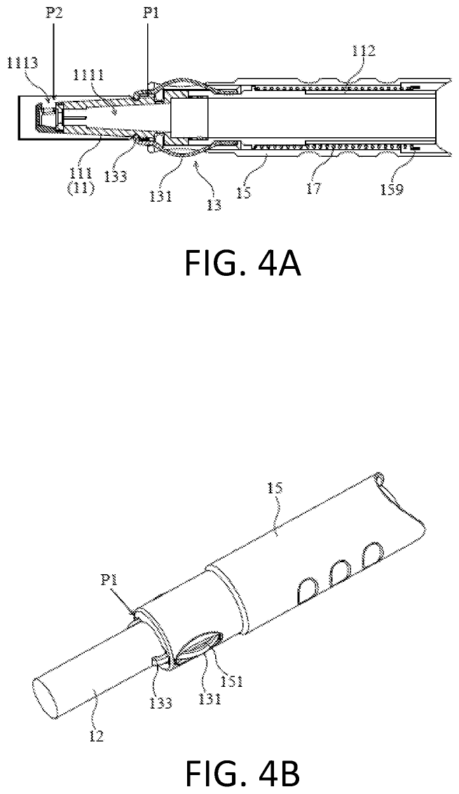

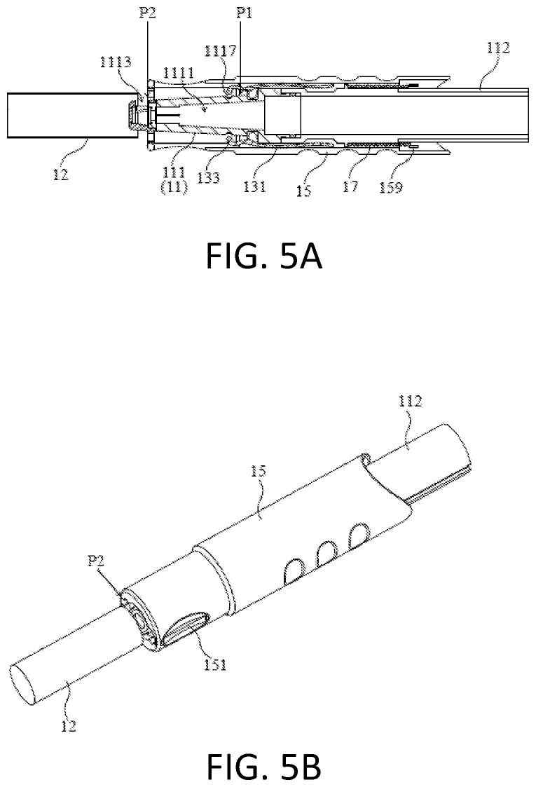

FIGS. 3A and 3B are, respectively, a sectional view and a schematic diagram of an occult blood testing device operated in a first position and with a sleeved filter unit according to an embodiment of the invention. FIGS. 4A and 4B are, respectively, a sectional view and a schematic diagram of an occult blood testing device operated in a first position and with a clipped filter unit according to one embodiment of the invention. FIGS. 5A and 5B are, respectively, a sectional view and a schematic diagram of an occult blood testing device operated in a second position to remove a filter unit according to one embodiment of the invention.

As shown in FIGS. 3A and 3B, the sleeve 15 of the occult blood testing device 10 is at the first position P1, and the user presses the push portion 131 of the clip 13 to force the clip portion 133 of the clip 13 move away from the surface of the probe unit 111 or the clip recess 117 and make a gap between the elastic clip 13 and the probe unit 111.

As shown in FIGS. 4A and 4B, the user sleeves a filter unit 12 over the probe unit 111 to a position where a part of the filter unit 12 is placed between the clip portion 133 of the elastic clip 13 and the surface of the probe unit 111 or the clip recess 1117. The user then releases the push portion 131 of the elastic clip 13 and the clip portion 133 of the elastic clip 13 swings or moves towards the surface of the probe unit 111 or the clip recesses 1117 such that the filter unit 12 is clipped and held between the elastic clip 13 and the probe unit 111.

The user inserts the probe unit 111 that has been disposed with the filter unit 12 into the test solution, allowing the test solution to pass through the filter unit 12 and enter the test space 1111 through the opening 1113 of the probe unit 111, wherein the solid residue in the test solution is isolated by the filter unit 12 and does not come in contact with the probe unit 111 or enter the test space 1111 of the probe unit 111.

After the occult blood testing has completed, as shown in FIGS. 5A and 5B, the user pushes the sleeve 15 in the direction of the front end of the probe unit 111 and the filter unit 12 and moves the sleeve 15 from the first position P1 to the second position P2. The inner surface of the sleeve 15 compresses the push portion 131 of the elastic clip 13, which in turn forces the clip portion 133 of the elastic clip 13 to leave and move away from the surface of the probe unit 111 or the clip recess 1117. The block portion 157 disposed on one end of the sleeve 15 pushes the filter unit 12 at the front end of the probe unit 111 to separate the filter unit 12 and the probe unit 111. After removing the filter unit 12, the user just needs to wash the probe unit 111 with running water to complete the cleaning of the occult blood testing device 10.

The above disclosure is only the preferred embodiment of the present invention, and not used for limiting the scope of the present invention. All equivalent variations and modifications on the basis of shapes, structures, features and spirits described in claims of the present invention should be included in the claims of the present invention.

What is claimed is:

1. An occult blood testing device comprising:
a hemoglobin sensor comprising:
a probe unit;
at least one light emitting unit for emitting at least one incident light beam, wherein the incident light beam is aimed towards a direction of the probe unit and at a test solution to produce at least one detection light beam; and
at least one light sensing unit for receiving the at least one detection light beam;
at least one elastic clip disposed on a surface of the hemoglobin sensor, wherein the elastic clip comprises a push portion and a clip portion, and the clip portion clips a filter unit to the hemoglobin sensor; and
a sleeve sleeved over the hemoglobin sensor and movable between a first position and a second position relative to the hemoglobin sensor, wherein when the sleeve is at the first position, the push portion of the elastic clip is not compressed and the clip portion of the elastic clip rests on the probe unit and clips the filter unit, and when the sleeve is at the second position, the push portion of the elastic clip is compressed and the clip portion of the elastic clip moves away from the probe unit and releases the filter unit.

2. The occult blood testing device of claim 1, wherein the sleeve comprises at least one through hole and the push portion of the elastic clip is positioned inside the through hole.

3. The occult blood testing device of claim 1, wherein the elastic clip comprises a lug, the probe unit comprises a plurality of connect recesses disposed on a surface thereof, and the lug of the elastic clip is positioned inside the connect recesses of the probe unit.

4. The occult blood testing device of claim 3, wherein the probe unit comprises at least one clip recess disposed on the surface thereof and the clip portion of the elastic clip is positioned inside the clip recess of the probe unit

5. The occult blood testing device of claim 4, wherein the elastic clip comprises an inner surface and an outer surface, the clip portion and the lug are disposed on the inner surface of the elastic clip and protrude therefrom, and the push portion is an arc structure curved in a direction from the inner surface to the outer surface.

6. The occult blood testing device of claim 3, wherein the elastic clip comprises a rotating axle disposed on the lug, the probe unit comprises a plurality of protrusions disposed on a side of the connect recess, and the rotating axle of the elastic clip is positioned between two of the protrusions.

7. The occult blood testing device of claim 1, further comprising an elastic unit disposed between the sleeve and the hemoglobin sensor, and the sleeve at the second position compresses the elastic unit.

8. The occult blood testing device of claim 7, wherein the hemoglobin sensor comprises an outer surface and a flange disposed on the outer surface, the sleeve comprises an inner surface and a protruding unit disposed on the inner surface, and one end of the elastic unit is connected to the flange of the hemoglobin sensor and another end of the elastic unit is connected to the protruding unit of the sleeve.

9. The occult blood testing device of claim 1, wherein the sleeve comprises an opening disposed at one end thereof, at least one recess disposed around the opening, and an accommodating space, wherein the opening and the recess are in fluid communication with the accommodating space in the sleeve, the probe unit is positioned inside the opening of the sleeve, and the elastic clip is positioned inside the recess of the sleeve.

10. The occult blood testing device of claim 1, wherein the probe unit comprises at least one opening and a test space, the opening is disposed at one end of the probe unit and fluidly connected to the test space.
